SEQUENCE LISTING (2 sequences)

SEQ ID NO: 1 - DNA, 1434 bp, Arabidopsis thaliana
  misc_feature: TIGR database F4I10_150
atgaaggaacgcaaatgttggttcgctgatttcagcggagatcgccggctgcagaatgcggtttccgattgccttgatctgcttgacttttcttcagaggagctcacttggtctgcttctgcttctgagaatccaaaaggtaagggcaacggaacaggggacgttggatcggacacaaggacatggttaagtgctgctctctccaaccaagccacttgtatggaaggcttcgatggtacctctggtctcgtaaaatcccttgtcgctggcagtctcgatcaactatattctatgcttagggaattgttaccacttgtgcaaccagagcagaaacccaaggctgtttctaaacctggccctattgctaaaggaccgaaggcaccacctggccgtaaactcagagacaccgacgaagacgaaagccttcaattcccagactgggtcagaccggacgatcgtaaactattagaatcaaacggtaggacttacgacgttagtgtagcattggatgggacaggcaacttcacaaagataatggacgccataaagaaagctcctgattatagctcgacacgtttcgtcatatacattaaaaaaggtttatatttggagaatgttgagatcaagaagaagaaatggaacattgtaatgttaggtgatggcattgacgtgactgttatttccggtaaccgcagcttcatcgatggttggaccactttccgatcagctacattcgcggtaagcggaagaggattcttagcaagagatataacgttccagaacacagcagggccagagaagcatcaggcggtagcactgagatcagactctgacctctctgtgttcttcagatgtgcgatgaggggttatcaagatacactctacacgcacaccatgcgtcagttctaccgtgagtgcaccatcacgggaacagtagattttatatttggggatgggactgttgtgtttcaaaactgtcaaattttggctaaaagaggactccctaaccaaaagaacaccatcactgcacaaggccggaaagatgtcaaccaaccctcaggattctcaattcaattcagcaatatctcggcggacgcagatttggtcccatacctaaacacgacaaggacgtatctaggaaggccatggaagctatattcgagaacggttttcattaggaacaatatgagcgacgttgtgagacccgaaggatggcttgagtggaacgctgattttgcgttggacacactcttctacggagagttcatgaactacgggcctggttcaggactcagcagccgagtcaaatggcctggttaccacgtctttaacaactcggatcaggctaacaacttcaccgtttctcagttcattaaaggaaatctttggctgccttctacgggagtaacgtttagtgatggcttgtatatctaa

SEQ ID NO: 2 - protein, 477 aa, Arabidopsis thaliana
MKERKCWFADFSGDRRLQNAVSDCLDLLDFSSEELTWSASASENPKGKGNGTGDVGSDTRTWLSAALSNQATCMEGFDGTSGLVKSLVAGSLDQLYSMLRELLPLVQPEQKPKAVSKPGPIAKGPKAPPGRKLRDTDEDESLQFPDWVRPDDRKLLESNGRTYDVSVALDGTGNFTKIMDAIKKAPDYSSTRFVIYIKKGLYLENVEIKKKKWNIVMLGDGIDVTVISGNRSFIDGWTTFRSATFAVSGRGFLARDITFQNTAGPEKHQAVALRSDSDLSVFFRCAMRGYQDTLYTHTMRQFYRECTITGTVDFIFGDGTVVFQNCQILAKRGLPNQKNTITAQGRKDVNQPSGFSIQFSNISADADLVPYLNTTRTYLGRPWKLYSRTVFIRNNMSDVVRPEGWLEWNADFALDTLFYGEFMNYGPGSGLSSRVKWPGYHVFNNSDQANNFTVSQFIKGNLWLPSTGVTFSDGLYI

(19) United States
(12) Patent Application Publication
(10) Pub. No.: US 2005/0112715 A1
(43) Pub. Date: May 26, 2005

(54) Methods for the identification of inhibitors of pectin esterase expression or activity in plants

(76) Inventors: Adel Zayed, Durham, NC (US); Robert Ascenzi, Cary, NC (US); Douglas Boyes, Chapel Hill, NC (US); Rao Mulpuri, Apex, NC (US); Neil Hoffman, Chapel Hill, NC (US); Keith Davis, Durham, NC (US); Jorn Gorlach, Manchester, NJ (US); Jeffrey Woessner, Hillsborough, NC (US); Carol Hamilton, Apex, NC (US); Kenneth Phillips, Ralegh, NC (US)

Correspondence Address: Icoria, Inc., 108 T.W. ALEXANDER DRIVE, P O BOX 14528, RTP, NC, 27709-4528, US

(21) Appl. No.: 10/717,879

(22) Filed: Nov. 20, 2003

(51) Int. Cl. C12Q 1/44; A01N 25/00

(52) U.S. Cl. 435/19; 504/116.1

(57) ABSTRACT

The present inventors have discovered that pectin esterase is essential for plant growth. Specifically, the inhibition of pectin esterase gene expression resulted in seedlings that were damaged, short, and stunted at a very early growth stage. Thus, pectin esterase can be used as a target for the identification of herbicides. Accordingly, the present invention provides methods for the identification of compounds that inhibit pectin esterase expression or activity, comprising: contacting a compound with a pectin esterase and detecting the presence and/or absence of binding between said compound and said pectin esterase, or detecting a decrease in pectin esterase expression or activity. The methods of the invention are useful for the identification of herbicides.

This application is the national phase under 35 U.S.C. § 371 of PCT International Application No. PCT/US02/16648, that has an International filing date of May 29, 2002, which designated the United States of America and which claims the benefit of U.S. Provisional Application Ser. No. 60/294,487, filed May 30, 2001.

### FIELD OF THE INVENTION

The invention relates generally to plant molecular biology. In particular, the invention relates to methods for the identification of herbicides.

### BACKGROUND OF THE INVENTION

Pectin esterase (PE; EC 3.1.1.11) is also known as pectinesterase, pectin methylesterase, pectin demethoxylase, and pectin methoxylase. PE catalyzes the demethylation of pectin, which is a major component of dicotyledonous plants' cell walls. The enzyme is widely distributed in plants and may have roles in the deposition of pectin in developing tissues and in the wall loosening and cell separation that occurs in cell expansion, fruit ripening and abscission.

Prokaryotic and eukaryotic pectin esterases share a few regions of sequence similarity. Two regions have been selected as signature patterns. The first is based on a region in the N-terminal section of these enzymes. It contains a conserved tyrosine which may play a role in the catalytic mechanism. The second pattern corresponds to the best-conserved region, an octapeptide located in the central part of these enzymes. It has been reported that the Arabidopsis genome contains at least twelve pectin methylesterase (PME)-related genes. Micheli et al. (1998) Gene 220:13-20. Whereas most of these genes appeared to be more or less ubiquitously expressed throughout the plant, several genes were distinguishable by their strikingly specific expression in certain organs.

Pectin esterase has been discussed at length in the literature because of its market potential in affecting fruit ripening and resistance to splitting, which could prove to be of great value to fruit growers and processors. It is also widely used in the food industry in processing fruits and vegetables. However, the prior art does not suggest the use of PE as a herbicide target.

### SUMMARY OF THE INVENTION

The present inventors have discovered that antisense expression of a pectin esterase cDNA in Arabidopsis caused significant developmental abnormalities during early development. Seedlings were damaged, shorter than controls, extremely stunted and did not progress beyond a very early stage of growth. Thus, the present inventors have discovered that pectin esterase is essential for normal seed development and growth, and can be used as a target for the identification of herbicides. Accordingly, the present invention provides methods for the identification of compounds that inhibit pectin esterase expression or activity, comprising: contacting a candidate compound with a pectin esterase and detecting the presence or absence of binding between said compound and said pectin esterase, or detecting a decrease in pectin esterase expression or activity. The methods of the invention are useful for the identification of herbicides.

### BRIEF DESCRIPTION OF THE DRAWINGS

FIG. 1 illustrates the biochemical pathway in which pectin esterase is active.

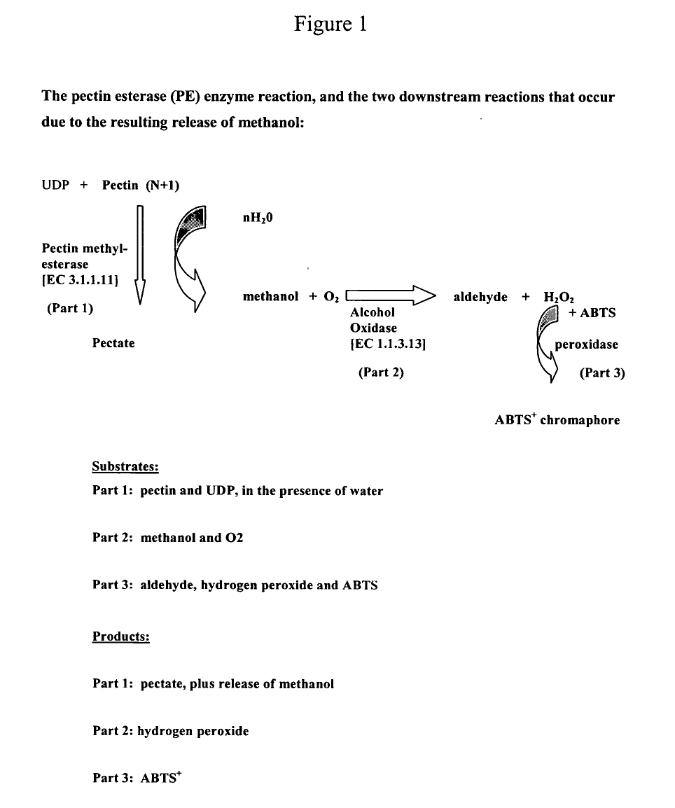

### DETAILED DESCRIPTION OF THE INVENTION

Definitions

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

The term “pectin” refers herein to poly (1,4-alpha-D-galacturonide).

The term “methanol” refers to the alcohol CH₃OH.

The term “ABTS” refers to 2,2′-azino-bis-(3-ethylbenzothiazoline-6-sulfonic acid). “ABTS⁺” represents the oxidized form of ABTS, and emits a color which can be monitored spectrophotometrically.

The term “hydrogen peroxide” refers to the compound H₂O₂.

The term “pectate” refers to poly(1,4-alpha-D-galacturonate).

The term “UDP” refers to uridine diphosphate.

As used herein, the term “pectin esterase” (EC 3.1.1.11) may be considered synonymous with PE, pectinesterase, pectin methylesterase, pectin demethoxylase, pectin methoxylase, and the pectin esterase-like protein represented herein as SEQ ID NO:2, and refers to any polypeptide or protein that catalyzes the demethylation of pectin to form pectate. The substrates for this reaction include UDP and pectin (in the presence of water), and the products include pectate, with a corresponding release of methanol.

The term “herbicide,” as used herein, refers to a compound that may be used to kill or suppress the growth of at least one plant, plant cell, plant tissue or seed.

The term “inhibitor,” as used herein, refers to a chemical substance that inactivates the enzymatic activity of pectin esterase. The inhibitor may function by interacting directly with the enzyme, a cofactor of the enzyme, the substrate of the enzyme, or any combination thereof.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

The “percent (%) sequence identity” between two polynucleotide or two polypeptide sequences is determined according to the either the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings) or using Smith Waterman Alignment (Smith and Waterman (1981) Adv Appl Math 2:482) as incorporated into GENEMATCHER PLUS (Paracel, Inc.) (using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

The term “cDNA” is synonymous with complementary deoxyribonucleic acid.

By “mRNA” is meant messenger ribonucleic acid.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

The term “specific binding” refers to an interaction between pectin esterase and a molecule or compound, wherein the interaction is dependent upon the primary amino acid sequence or the conformation of pectin esterase.

### EMBODIMENTS OF THE INVENTION

The present inventors have discovered that inhibition of a pectin esterase gene expression resulted in damaged, short and extremely stunted seedlings that did not progress beyond a very early growth stage. Thus, the inventors have demonstrated that pectin esterase is a target for herbicides.

Accordingly, the invention provides methods for identifying compounds that inhibit pectin esterase gene expression or activity. Such methods include ligand binding assays, assays for enzyme activity and assays for pectin esterase gene expression. Any compound that is a ligand for pectin esterase other than its substrates may have herbicidal activity. For the purposes of the invention, “ligand” refers to a molecule that will bind to a site on a polypeptide. The compounds identified by the methods of the invention are useful as herbicides.

Thus, in one embodiment, the invention provides a method for identifying a compound as a candidate for a herbicide, comprising:
a) contacting a pectin esterase with said compound; and
b) detecting the presence and/or absence of binding between said compound and said pectin esterase, wherein binding indicates that said compound is a candidate for a herbicide.

By “pectin esterase” is meant any enzyme (i.e., polypeptide or protein) that catalyzes the demethylation of pectin to form pectate. The pectin esterase may have the amino acid sequence of a naturally occuring pectin esterase found in a plant, animal or microorganism, or may have an amino acid sequence derived from a naturally occuring sequence. Preferably the pectin esterase is a plant pectin esterase.

By “plant pectin esterase” is meant an enzyme that can be found in at least one plant, and which catalyzes the demethylation of pectin to form pectate. The pectin esterase may be from any plant, including both monocots and dicots.

In one embodiment, the pectin esterase is an Arabidopsis pectin esterase. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis pectin esterase is from Arabidopsis thaliana.

The cDNA of SEQ ID NO:1 and amino acid sequence of SEQ ID NO:2 of the Arabidopsis thaliana pectin esterase-like protein can be found herein as well as in the TIGR database at accession No. F4I10^{—}150. This pectin esterase cDNA sequence may be used as a probe to isolate pectin esterase cDNAs or genes from additional organisms, and to synthesize pectin esterase polypeptides.

In various embodiments, the pectin esterase is from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of a pectin esterase polypeptide may be used in the methods of the invention. The fragments comprise at least 10 consecutive amino acids of a pectin esterase. Preferably, the fragment comprises at least 15, 20, 25, 30, 35, 40, 50, 60, 70, 80, 90 or at least 100 consecutive amino acids residues of a pectin esterase. In one embodiment, the fragment is from an Arabidopsis pectin esterase.

Polypeptides having at least 80% sequence identity with a plant pectin esterase are also useful in the methods of the invention. Preferably, the sequence identity is at least 85%, more preferably the identity is at least 90%, most preferably the sequence identity is at least 95%.

In addition, it is preferred that the polypeptide has at least 50% of the activity of a plant pectin esterase. More preferably, the polypeptide has at least 60%, at least 70%, at least 80% or at least 90% of the activity of a plant pectin esterase. Most preferably, the polypeptide has at least 50%, at least 60%, at least 70%, at least 80% or at least 90% of the activity of the A. thaliana pectin esterase protein.

Thus, in another embodiment, the invention provides a method for identifying a compound as a candidate for a herbicide, comprising:
a) contacting said compound with at least one polypeptide selected from the group consisting of: a plant pectin esterase, a polypeptide comprising at least ten consecutive amino acids of a plant pectin esterase, a polypeptide having at least 85% sequence identity with a plant pectin esterase, and a polypeptide having at least 80% sequence identity with a plant pectin esterase and at least 50% of the activity thereof; and
b) detecting the presence and/or absence of binding between said compound and said polypeptide, wherein binding indicates that said compound is a candidate for a herbicide.

Pectin esterase activity refers to an enzyme (i.e., protein or polypeptide) which catalyzes the demethylation of pectin to form pectate. Methods for measuring pectin esterase activity are known in the art. See, for example, Mango and Haas (1997) Analytical Biochemistry 244:357-66.

Any technique for detecting the binding of a ligand to its target may be used in the methods of the invention. Preferably, the ligand and target are combined in a buffer. In one embodiment, the buffer is 100 mM sodium phosphate, pH 8.5. In addition, polypeptides and proteins that can reduce non-specific binding, such as BSA, or protein extracts from cells that do not produce the target, may be included in a binding assay.

Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a pectin esterase protein or a fragment or variant thereof, the unbound protein is removed and the bound pectin esterase is detected. In a preferred embodiment, bound pectin esterase is detected using a labeled binding partner, such as a labeled antibody. In a variation of this assay, pectin esterase is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods. In one embodiment, the binding of a compound to pectin esterase is by transferred nuclear overhause effect spectroscopy (TRNOESY). See Kennedy et al. (1999) J Enzyme Inhib 14:217-237.

Once a compound is identified as a candidate for a herbicide, it can be tested for the ability to inhibit pectin esterase enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with said herbicide candidate and detecting the presence or absence of a decrease in the growth or viability of said plant or plant cells.

By decrease in growth, is meant that the herbicide candidate causes at least a 10% decrease in the growth of the plant or plant cells, as compared to the growth of the plants or plant cells in the absence of the herbicide candidate. By a decrease in viability is meant that at least 20% of the plants cells, or portion of the plant contacted with the herbicide candidate are nonviable. Preferably, the growth or viability will be at decreased by at least 40%. More preferably, the growth or viability will be decreased by at least 50%, 75% or at least 90% or more. Methods for measuring plant growth and cell viability are known to those skilled in the art. It is possible that a candidate compound may have herbicidal activity only for certain plants or certain plant species.

The ability of a compound to inhibit pectin esterase activity can be detected using in vitro enzymatic assays in which the disappearance of a substrate or the appearance of a product is directly or indirectly detected. Pectin esterase catalyzes the reaction of UDP (uridine diphosphate) plus pectin, with water as a co-factor, to the corresponding pectate with methanol. The methanol, with O₂ and alcohol oxidase produces hydrogen peroxide. The hydrogen peroxide, with aldehyde, ABTS, and peroxidase, releases ABTS+. Methods for detection of UDP, pectin, pectate, methanol, hydrogen peroxide and ABTS⁺ include spectrophotometry, mass spectroscopy, thin layer chromatography (TLC) and reverse phase HPLC.

Thus, the invention provides a method for identifying a compound as a candidate for a herbicide, comprising:
a) contacting a UDP and pectin with PE, in the presence of water;
b) contacting said UDP and pectin with PE and said compound, in the presence of water; and
c) contacting the methanol resulting from steps a) and b) with O₂ and alcohol oxidase;
d) contacting the hydrogen peroxide resulting from step c) with aldehyde, ABTS and peroxidase; and
e) determining the concentration of at least one of UDP, pectin, pectate, methanol, hydrogen peroxide and/or ABTS⁺ after the contacting of any of steps a), b), c) and/or d).

If a candidate compound inhibits PE activity, a higher concentration of the substrates (pectin and UDP) and a lower level of the products (pectate, methanol, hydrogen peroxide and ABTS⁺) will be detected in the presence of the candidate compound (step b) than in the absence of the compound (step a).

Preferably the pectin esterase is a plant pectin esterase. Enzymatically active fragments of a plant pectin esterase are also useful in the methods of the invention. For example, a polypeptide comprising at least 100 consecutive amino acid residues of a plant pectin esterase may be used in the methods of the invention. In addition, a polypeptide having at least 80%, 85%, 90%, 95%, 98% or at least 99% sequence identity with a plant pectin esterase may be used in the methods of the invention. Preferably, the polypeptide has at least 80% sequence identity with a plant pectin esterase and at least 50%, 75%, 90% or at least 95% of the activity thereof.

Thus, the invention provides a method for identifying a compound as a candidate for a herbicide, comprising:
a) contacting a UDP and pectin, in the presence of water, with a polypeptide selected from the group consisting of: a polypeptide having at least 85% sequence identity with a plant PE, a polypeptide having at least 80% sequence identity with a plant PE and at least 50% of the activity thereof, and a polypeptide comprising at least 100 consecutive amino acids of a plant PE;
b) contacting said UDP and pectin with said polypeptide and said compound, in the presence of water; and
c) contacting the methanol resulting from steps (a) and (b) with O₂ and alcohol oxidase;
d) contacting the hydrogen peroxide resulting from step (c) with aldehyde, ABTS and peroxidase; and
e) determining the concentration of at least one of UDP, pectin, pectate, methanol, hydrogen peroxide and/or ABTS⁺ after the contacting of any of steps (a), (b), (c) and/or (d).

Again, if a candidate compound inhibits pectin esterase activity, a higher concentration of the substrates (UDP and pectin) and a lower level of the products (pectate, methanol, hydrogen peroxide and ABTS⁺) will be detected in the presence of the candidate compound (step b) than in the absence of the compound (step a).

For the in vitro enzymatic assays, pectin esterase protein and derivatives thereof may be purified from a plant or may be recombinantly produced in and purified from a plant, bacteria, or eukaryotic cell culture. Preferably these proteins are produced using a baculovirus or E. coli expression system. A method for the purification of pectin esterase is described in Ding et al. (2000) J Agric Food Chem 48:3052-7. Other methods for the purification of pectin esterase proteins and polypeptides are known to those skilled in the art.

As an alternative to in vitro assays, the invention also provides plant and plant cell based assays. In one embodiment, the invention provides a method for identifying a compound as a candidate for a herbicide, comprising:
a) measuring the expression of pectin esterase in a plant or plant cell in the absence of said compound;
b) contacting a plant or plant cell with said compound and measuring the expression of pectin esterase in said plant or plant cell;
c) comparing the expression of pectin esterase in steps (a) and (b).

A reduction in pectin esterase expression indicates that the compound is a herbicide candidate. In one embodiment, the plant or plant cell is an Arabidopsis thaliana plant or plant cell.

Expression of pectin esterase can be measured by detecting pectin esterase primary transcript or mRNA, pectin esterase polypeptide or pectin esterase enzymatic activity. Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Methods for detecting pectin esterase RNA include, but are not limited to amplification assays such as quantitative PCR, and/or hybridization assays such as Northern analysis, dot blots, slot blots, in-situ hybridization, transcriptional fusions using a pectin esterase promoter fused to a reporter gene, bDNA assays and microarray assays.

Methods for detecting protein expression include, but are not limited to, immunodetection methods such as Western blots, His Tag and ELISA assays, polyacrylamide gel electrophoresis, mass spectroscopy and enzymatic assays. Also, any reporter gene system may be used to detect pectin esterase protein expression. For detection using gene reporter systems, a polynucleotide encoding a reporter protein is fused in frame with pectin esterase, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2:1104; Prost et al. (1986) Gene 45:107-111), β-galactosidase (Nolan et al. (1988) Proc Natl Acad Sci USA 85:2603-2607), alkaline phosphatase (Berger et al. (1988) Gene 66:10), luciferase (De Wet et al. (1987) Mol Cell Biol 7:725-737), β-glucuronidase (GUS), fluorescent proteins, chromogenic proteins and the like. Methods for detecting pectin esterase activity are described above.

Chemicals, compounds or compositions identified by the above methods as modulators of pectin esterase expression or activity can then be used to control plant growth. For example, compounds that inhibit plant growth can be applied to a plant or expressed in a plant, in order to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants were grown Scotts Metro-Mix™ soil (the Scotts Company) or a similar soil mixture in an environmental growth room at 22° C., 65% humidity, 65% humidity and a light intensity of ˜100 μ-E m⁻²s⁻¹ supplied over 16 hour day period.

Seed Sterilization

All seeds were surface sterilized before sowing onto phytagel plates using the following protocol.
1. Place approximately 20-30 seeds into a labeled 1.5 ml conical screw cap tube. Perform all remaining steps in a sterile hood using sterile technique.
2. Fill each tube with 1 ml 70% ethanol and place on rotisserie for 5 minutes.
3. Carefully remove ethanol from each tube using a sterile plastic dropper; avoid removing any seeds.
4. Fill each tube with 1 ml of 30% Clorox and 0.5% SDS solution and place on rotisserie for 10 minutes.
5. Carefully remove bleach/SDS solution.
6. Fill each tube with 1 mL sterile dI H₂O; seeds should be stirred up by pipetting of water into tube. Carefully remove water. Repeat 3 to 5 times to ensure removal of Clorox/SDS solution.
7. Fill each tube with enough sterile dI H₂O for seed plating (˜200-400 μl ). Cap tube until ready to begin seed plating.
   Plate Growth Assays

Surface sterilized seeds were sown onto plate containing 40 ml half strength sterile MS (Murashige and Skoog, no sucrose) medium and 1% Phytagel using the following protocol:
1. Using pipette man and 200 μl tip, carefully fill tip with seeds and 0.1% agarose solution. Place 10 seeds across the top of the plate, about ¼ in down from the top edge of the plate.
2. Place plate lid ¾ of the way over the plate and allow to dry for 30 minutes or until agarose solution is dry. It is important to allow agarose solution to dry completely before sealing up plates in order to prevent contamination.
3. Using sterile micropore tape, seal the edge of the plate where the top and bottom meet.
4. Place plates stored in a vertical rack in the dark at 4° C. for three days.
5. Three days after sowing, the plates transferred into a growth chamber with a day and night temperature of 22 and 20° C., respectively, 65% humidity and a light intensity of ˜100 μ-E m⁻²s⁻¹ supplied over 16 hour day period.
6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant Expressing the Driver

The “Driver” is an artificial transcription factor comprising a chimera of the DNA-binding domain of the yeast GAL4 protein (amino acid residues 1-137) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of Pectin Esterase Antisense Expression Cassettes in a Binary Vector

A fragment of an Arabidopsis thaliana pectin esterase cDNA corresponding to TIGR accession No. F4I10_{—}150 was ligated into the PacI/AscI sites of an E. coli/Agrobacterium binary vector in the antisense orientation. This placed transcription of the pectin esterase antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E.coli. Kanamycin resistant clones were selected and purified. DNA was isolated from each clone and characterized by PCR and sequence analysis. pPG774 expresses the A. thaliana pectin esterase antisense RNA corresponding to the cDNA (complementary deoxyribonucleic acid) shown in SEQ ID NO:1, which encodes the pectin esterase-like polypeptide sequence shown in SEQ ID NO:2. The name pPG774 is used for applicants' internal reference, and one skilled in the art will recognize that this particular plasmid is not required to practice the present invention. Any other suitable type of plasmid may be used to express antisense RNA complementary to a portion of the cDNA of SEQ ID NO:1, including plasmids similar to the types found in U.S. Pat. Nos. 5,107,065 and 5,254,800, incorporated herein by reference.

The antisense expression cassette and a constitutive barnase expression cassette are located between right and left T-DNA borders. Thus, the antisense expression cassettes can be transferred into a recipient plant cell by agroinfection.

### EXAMPLE 3

### Transformation of Agrobacterium with the Target Expression Cassette

pPG774 was transformed into Agrobacterium tumefaciens by electroporation. Transformed Agrobacterium colonies were isolated using Basta selection. DNA was prepared from purified Basta resistant colonies and the inserts were amplified by PCR and sequenced to confirm sequence and orientation.

### EXAMPLE 4

### Construction of Arabidopsis Pectin Esterase Antisense Target Plants

The pectin esterase target expression cassette was introduced into Arabidopsis thaliana wild-type plants by the following method. Five days prior to agroinfection, the primary inflorescence of Arabidopsis thaliana plants grown in 2.5 inch pots were clipped in order enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying pPG774. The culture was incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L SILWET L-77 (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then cover with a tall clear plastic dome in order to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected using glufosinate treatment. Approximately 70 mg seeds from an agrotransformed plant were mixed approximately 4:1 with sand and placed in a 2 ml screw cap cryo vial.

The surface of the seeds was sterilized using the chlorine gas method. Briefly, the open vials were placed in a vacuum desiccator in a safety hood. A glass beaker containing 200 ml 5.25% sodium hypochlorite solution was placed in the desiccator. Two ml concentrated HCl was added to the hypochlorite solution and the cover was placed on the desiccator. Vacuum was applied briefly to seal the dessicator, and the seeds were left in the desiccator overnight.

One vial of sterilized seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a 1:3000 dilution of LIBERTY (AgrEvo; 11.3% glufosinate) in water, 0.005% SILWET (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represent stably transformed T1 plants. Mature T1 plants are then dried and harvested for T2 seeds.

### EXAMPLE 5

### Effect of Pectin Esterase Antisense Expression in Arabidopsis Seedlings

The pectin esterase target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected over this 2-week period for growth and development. During this period, half of the seedlings (five of ten) derived from the pPG774 pectin esterase antisense target line S16624 were damaged, short and extremely stunted and did not progress beyond a very early growth stage. Thus, pectin esterase is essential for normal plant growth and development.

### EXAMPLE 6

### Assays for Inhibitors of Pectin Esterase Activity

The following assay may be used to determine pectin esterase enzyme activity. A 2× assay buffer containing 200 mM Tris pH 7.5, 200 mM NaCl, 0.25-0.5% pectin, alcohol oxidase, peroxidase and ABTS is made, and 50 ul is added to the protein extract of interest. A 50-ul aliquot of pectin esterase is added to the 2× assay buffer and incubated at room temperature for one hour. The reaction proceeds so that methanol is released from pectin by the pectin esterase. The methanol is then oxidized by the alcohol oxidase to form hydrogen peroxide. At this point in the reaction, the peroxidase catalyzes the reaction of hydrogen peroxide with formaldehyde and ABTS, to form an ABTS⁺ chromaphore. The optical density of the ABTS⁺ chromaphore solution is then read at a wavelength of 420 nm. This assay is described by Mango and Haas (1997) Analytical Biochem 244:357-66.

Alternatively, the activity of pectin esterase can be measured titrimetrically by following the increase in free carboxyl groups. The carboxyl groups released by PE from 0.25% pectin in the presence of 150 mM NaCl are titrated with 10 mM NaOH under N₂ while maintaining the pH at chosen values ranging from 5.2 to 8.4 with an automatic titrator such as the TTT 80 from Radiometer, Copenhagen, Denmark. Quick screening of pectin esterase activity after chromatographic fractionation is performed as follows: 20 ul of each protein fraction of interest is dispersed into enzyme-linked immunosorbent assay (ELISA) microwells containing 200 ul of a mixture composed of 0.5% pectin, 0.2M NaCl and 0.02% pH indicator (methyl red). Pectin esterase activity is then determined by identifying the wells in which the mixture turns from yellow (pH 7) to pink due to the release of H⁺. Guglielmino et al. (1997) Planta 202:70-5.

Further alternatives exist for monitoring the activity of the pectin esterase enzyme, including those methods described in U.S. Pat. Nos. 4,200,694 and 5,707,847, the contents of which are incorporated herein by reference.

While the foregoing describes certain embodiments of the invention, it will be understood by those skilled in the art that variations and modifications may be made and still fall within the scope of the invention.

1. A method for identifying a compound as a candidate for a herbicide, comprising:
a) contacting a pectin esterase with a compound; and
b) detecting the presence and/or absence of binding between said compound and said pectin esterase, wherein binding indicates that said compound is a candidate for a herbicide.

2. The method of claim 1, wherein said pectin esterase is a plant pectin esterase.

3. The method of claim 2, wherein said pectin esterase is an Arabidopsis pectin esterase.

4. A method for determining whether a compound identified as a herbicide candidate by the method of claim 1 has herbicidal activity, comprising: contacting a plant or plant cells with said herbicide candidate and detecting the presence or absence of a decrease in growth or viability of said plant or plant cells.

5. A method for identifying a compound as a candidate for a herbicide, comprising:
a) contacting a compound with at least one polypeptide selected from the group consisting of: an amino acid sequence comprising at least ten consecutive amino acids of a plant pectin esterase, an amino acid sequence having at least 85% sequence identity with a plant pectin esterase, and an amino acid sequence having at least 80% sequence identity with a plant pectin esterase and at least 50% of the activity thereof; and
b) detecting the presence and/or absence of binding between said compound and said polypeptide, wherein binding indicates that said compound is a candidate for a herbicide.

6. A method for determining whether a compound identified as a herbicide candidate by the method of claim 5 has herbicidal activity, comprising: contacting a plant or plant cells with said herbicide candidate and detecting the presence or absence of a decrease in growth or viability of said plant or plant cells.

7. A method for identifying a compound as a candidate for a herbicide, comprising:
a) contacting a UDP and pectin with pectin esterase, in the presence of water;
b) contacting said UDP and pectin with pectin esterase and a compound, in the presence of water;
c) contacting the methanol resulting from steps a) and b) with O₂ and alcohol oxidase;
d) contacting the hydrogen peroxide resulting from step c) with aldehyde, ABTS and peroxidase; and
e) determining the concentration of at least one of UDP, pectin, pectate, methanol, hydrogen peroxide and/or ABTS⁺ after the contacting of any of steps a), b), c) and/or d).

8. The method of claim 7, wherein said pectin esterase is a plant pectin esterase.

9. The method of claim 8, wherein said pectin esterase is an Arabidopsis pectin esterase.

10. A method for identifying a compound as a candidate for a herbicide, comprising:
a) contacting, in the presence of water, a UDP and pectin with a polypeptide selected from the group consisting of: a polypeptide having at least 85% sequence identity with a plant pectin esterase, a polypeptide having at least 80% sequence identity with a plant pectin esterase and at least 50% of the activity thereof, and a polypeptide comprising at least 100 consecutive amino acids of a plant pectin esterase;
b) contacting, in the presence of water, said UDP and pectin with said polypeptide and said compound; and
c) contacting the methanol resulting from steps a) and b) with O₂ and alcohol oxidase;
d) contacting the hydrogen peroxide resulting from step c) with aldehyde, ABTS and peroxidase; and
e) determining the concentration of at least one of UDP, pectin, pectate, methanol, hydrogen peroxide and/or ABTS⁺ after the contacting of any of steps a), b), c) and/or d).

11. A method for identifying a compound as a candidate for a herbicide, comprising:
a) measuring the expression of a pectin esterase in a plant or plant cell in the absence of a compound;
b) contacting a plant or plant cell with said compound and measuring the expression of said pectin esterase in said plant or plant cell;
c) comparing the expression of pectin esterase in steps (a) and (b).

12. The method of claim 11 wherein said plant or plant cell is an Arabidopsis plant or plant cell.

13. The method of claim 11, wherein the expression of pectin esterase is measured by detecting pectin esterase mRNA.

14. The method of claim 1 1, wherein the expression of pectin esterase is measured by detecting pectin esterase polypeptide.
